(19) United States
(12) Patent Application Publication
(10) Pub. No.: US 2019/0343926 A1
(43) Pub. Date: Nov. 14, 2019

(54) Microburst Insulin Infusion Restores Carbohydrate Metabolism

(71) Applicant: G. Ford Gilbert, McClellan, CA (US)

(72) Inventor: G. Ford Gilbert, McClellan, CA (US)

(21) Appl. No.: 15/823,564

(22) Filed: Nov. 27, 2017

(51) Int. Cl. A61K 38/28; A61K 31/7004; A61P 3/08; A23L 33/00

(52) U.S. Cl. CPC A61K 38/28; A61K 31/7004; A23V 2002/00; A23L 33/30; A61P 3/08

(57) ABSTRACT

A method for delivering glucose and insulin to a subject to improve carbohydrate metabolism. It is desirable to improve liver storage of glycogen, and in general to reduce excess fatty tissue in the liver. The method delivers a large amount of glucose accompanied by a series of bursts of insulin to the subject over a period of time. The amount of insulin in each burst, the interval between bursts and the amount of time to deliver each burst to the subject are selected so that the cellular processing of glucose is improved in the subject. The glucose is delivered in dose portions over an extended period of hours, typically providing a roughly even rate of glucose input during the insulin administration. Treatments are repeated over multiple days. For most subjects, an improvement in resting amount of carbohydrate metabolism is seen.

### FIELD OF THE INVENTION

This invention relates to medical treatments, and more specifically to a method of treating various diseases and conditions through administering a substantial caloric load coupled with administration of hormone to stimulate the body to convert that caloric load and increase metabolism in at least some cells. Body functioning converts intracellular adenosine triphosphate (ATP) as a part of normal cell function. In a human, complex control systems balance caloric inputs by managing internal hormone levels to consume, store, or eliminate various types of caloric inputs. This invention stimulates these systems to increase metabolism of cells within the body and thereby treat various diseases and conditions. Many patients find their metabolism after treatment to be in better balance, and the patient's health improved.

This invention relates to a method for delivering glucose and insulin to a subject to improve carbohydrate metabolism. It is desirable to improve liver storage of glycogen, and in general to reduce excess fatty tissue in the liver. The method delivers a large amount of glucose accompanied by a series of bursts of insulin to the subject over a period of time. The amount of insulin in each burst, the interval between bursts and the amount of time to deliver each burst to the subject are selected so that the cellular processing of glucose is improved in the subject. The glucose is delivered in dose portions over an extended period of hours, typically providing a roughly even rate of glucose input. Treatments are repeated over multiple days. For most subjects, an improvement in resting amount of carbohydrate metabolism is seen. There is a goal of restoring normal glucose processing in the individual.

### BACKGROUND

Glucose is the primary form of energy source used by human cells, and also for many other forms of life. Glucose uptake by cells is strongly influenced by circulating insulin levels. In general, when glucose levels rise, such as after a meal or otherwise taking in glucose, a well-functioning body produces insulin, usually in bursts. When the endogenous systems are out of balance and not functioning well, it can be helpful to add additional sugar and insulin in a balanced way to induce cells to take up significant quantities of glucose. The present invention is directed to particularly accessible sugar forms and adding insulin in patterns modeled on natural human patterns.

A primary thrust of the present invention is to provide a method and mechanism for adding a sugar substance, such as glucose, and a peptide hormone, such as insulin, in amounts and patterns that have been found to stimulate cellular activity. Among the many benefits of this stimulation are a variety of health benefits. Informally, it is easy to think of this method as “jump starting” the cells. Importantly, a goal of this invention is to restore or maintain typical body liver function and blood glucose management.

Many have studied the interaction of glucose and insulin in the human body. Much is understood, but this understanding is not yet comprehensive, particularly when looking at individual responses of individual subjects. The form and amount of energy supply (meal substitute), hormonal choice and stimulation patterns (amount, frequency, totals), monitoring to assess treatment progress and more are now better understood. This specification will detail useful and novel parameters.

In the treatment method of the invention, a substantial glucose input is taken, raising blood glucose levels significantly. This is followed by rapid and significant insulin bursts, intended to stimulate cells to take in this blood glucose. We expect cells to process such intracellular glucose. For insulin resistant cells, the repeated insulin bursts will challenge the cell repeatedly to respond to the signals to take in that glucose. As more cells do indeed take up glucose, we see glucose utilization increasing. The insulin burst amounts can be decreased as cell health improves over repeated treatment cycles. Using a fast acting insulin is advantageous.

A cell type of particular interest is the liver and liver cells involved in processing glucose for storage and normal body functions.

In the treatment method of the invention, the subject is given a carbohydrate energy drink, then an insulin burst sequence for a period of time. This is followed by a rest period, then a second drink is provided, together with a second insulin sequence. In the usual treatment sequence, a second rest period is followed by a third drink and third insulin sequence. This can be repeated with yet another drink and insulin sequence, although that is not the usual practice of the present invention. The subject's metabolic state is assessed before the first energy drink, after the series of drink and insulin administrations for the day, and at some point during the sequence of the day, typically but not necessarily about half way through series planned for the day.

There is a larger time cycle where this daily sequence is repeated on subsequent days. Depending on the metabolic state before and after a daily sequence, a next treatment might be scheduled for the next day, or after a break of a small or a larger number of days. It is not unusual for a person in poor balance to be treated two or three times in the first week, and similarly in the following week, then if progress is typical, the time between treatments will be increased to more days. When balance appears good, treatments might be repeated only every two or three weeks, and ultimately only rarely.

For perspective on the present invention, it is useful to have in mind well known details of human metabolism, carbohydrate consumption and processing, blood sugar changes after meals, and the impact of insulin and glucagon, among other physiological components and responses.

Referring to FIG. 1, from an article by J. Li et al., Journal of Theoretical Biology, 242 (2006), 722-735, this illustrates some of the major signaling hormones and flows that are impacted by the present invention. Near the top left of the figure, glucose is derived from exogenous sources in the form of glucose (oral or IV or other), a meal, or other. Internal glucose can derive from release of internal stores of glycogen in the liver (among other reserves). The level of glucose stimulates the pancreas to release insulin if glucose is too high, and glucagon if glucose is too low. These hormones stimulate the liver to slow glucose release (increase storage), or to increase release (decrease storage), all intended to maintain glucose within physiologically acceptable ranges in the body. In general, these changes are not instantaneous but rather nudge changes incrementally over some time. We know from separate studies that insulin is released from the pancreas in microbursts.

Referring to FIG. 2, from github-dot-com/Open-Systems-Pharmacology/Glucose-Insulin-Model, we see a model of glucose insulin interaction for a Type 1 diabetes patient, i.e. not fully normal human physiology.

“The model is provided as a ready-to-use MoBi project “GlucoseInsulinModel.mbp3” . . . . The distributed project file includes six exemplary simulations of different perturbation experiments. Experimental conditions and data are reported in [S, R] Following protocols are simulated:
Intravenous glucose tolerance test (IVGTT): 500 mg/kg body weight glucose infused intravenously over 3 minutes [S]
Intravenous insulin tolerance test (IVITT): 0.04 IU/kg body weight insulin infused intravenously over 3 minutes [S]
Continuous insulin infusion (CIVI): 0.25 mIU/kg body weight/min insulin infusion for 150 minutes [S]
Oral glucose tolerance test (OGTT): Ingestion of 100 g glucose solution [S]
(two other examples not cited in the present patent application)
S: Sorensen J T. A physiologic model of glucose metabolism in man and its use to design and assess improved insulin therapies for diabetes. Thesis (Sc. D.). Massachusetts Institute of Technology (1985).
R: Regittnig W, Trajanoski Z, Leis H J, Ellmerer M, Wutte A, Sendlhofer G, Schaupp L, Brunner G A, Wach P, Pieber T R. Plasma and interstitial glucose dynamics after intravenous glucose injection: evaluation of the single-compartment glucose distribution assumption in the minimal models. Diabetes (1999), 48(5), 1070-1081.

### (End of Git Hub Reference)

Looking in particular at the IVITT intravenous insulin challenge, this is 40 mU/kg over 3 minutes, or 13 mU/kg/minute. Various of the charts show steady state resting insulin blood levels on the order of 25-50 pmol/L. In the tolerance test, levels go to some 100 times this. Note in particular the rapid drop after the infusion is stopped. This drop is on the order of 1.7 minutes to drop from 3000 to 1500 (a half life), slowing to more like 3 minutes to drop from 1000 to 500 (reaches 500 at 10 minutes, still some 10-20× baseline). Glucose levels drop by almost half over about 30 minutes (a slow response), and rebuild over about three times that time frame, mostly restored by 120 minutes. This is a hypoglycemic level, but brief and presumably under medical attention.

Comparing the CIVII continuous IV insulin infusion, with a much smaller 0.25 mU/kg, continuously. This is 2% of the smallest level used in the present invention at a momentary level, but cumulatively in the general range of insulin released over a normal day. Compare 0.25 mU/kg for 40 minutes=10 mU/kg with a similar 10 mU/kg/burst in the present invention. FIG. 5 shows 15 mU/kg/hr, with an insulin level reaching about three times the baseline level, after some 20 minutes of initial consumption by the body as the glucose levels begin to taper off. As glucose is falling from about 25 minutes to 90 minutes, from baseline 4 to about 3 mmol/L (72 to about 50 mg/dl, dangerously low if continued too long). The continued infusion shows a steady state with insulin increased about 3 times and glucose decreased about 25%. Presumably this is depleting body stores of glycogen to maintain this balance.

Looking at the OGTT Oral Glucose Tolerance Test, taking 100 g of glucose (a relatively large amount for this test, although the patient size is not reported), blood glucose levels increase by about 75% from a resting about 4 mmol/L (72 mg/dl) to about 7 mmol/L (125 mg/dl), again in about 30 minutes. Insulin levels rise in the same time frame, then hold steady for the next some 45 minutes, as the glucose level starts to decrease. Glucose returns to the original level after about 150 minutes total, while insulin continues to decrease, reaching baseline levels after about 250 minutes, more than 4 hours. The glucose level dips some from 150 to 250 minutes, then rises a bit, presumably from glucagon stimulation and glycogen release replenishing the sugar levels.

Note that these are all multiparametric environments, as various body processes, reservoirs, inputs, and control systems are involved. End of Github discussion.

### Cellular Level

Looking at a more cellular and molecular level, very briefly, carbohydrates from food are converted to glucose. The body is designed to move much of ingested glucose directly into cells. The body moves some of the glucose into glycogen, particularly in the liver. Glycogen is used during fasting periods as a continuing source of glucose for the cells of the body. Within cells, glucose is converted to pyruvate, with a net buildup of stored energy as ATP and NADH. Pyruvate is a key input to the tricarboxylic acid cycle (TCA or Krebs cycle). This is a primary engine of cell metabolism. ATP is consumed in many parts of the cell. This is particularly important in mitochondria. Mitochondria can be considered the power generators of cells.

Well functioning cells are important for general health. While this may seem a simple truism, the larger the number of well functioning cells, the better chance for the subject to be in good health.

Evolution has organized metabolism into a complex of pathways, and alternate pathways, with the goal of keeping cells energized and performing their diverse individual and collective functions. There are many ways for this complex to get out of balance. This is often expressed as disease, or the result of disease. This provides strong motivation for us to understand normal systems. We seek ways to correct imbalances as and when we can.

Metabolism has been studied for centuries. From the Wikipedia entry: “Metabolism is usually divided into two categories: catabolism, the breaking down of organic matter for example, the breaking down of glucose to pyruvate, by cellular respiration, and anabolism, the building up of components of cells such as proteins and nucleic acids. Usually, breaking down releases energy and building up consumes energy.

“The chemical reactions of metabolism are organized into metabolic pathways, in which one chemical is transformed through a series of steps into another chemical, by a sequence of enzymes. Enzymes are crucial to metabolism because they allow organisms to drive desirable reactions that require energy that will not occur by themselves, by coupling them to spontaneous reactions that release energy. Enzymes act as catalysts that allow the reactions to proceed more rapidly. Enzymes also allow the regulation of metabolic pathways in response to changes in the cell's environment or to signals from other cells.

“The metabolic system of a particular organism determines which substances it will find nutritious and which poisonous . . . . The speed of metabolism, the metabolic rate, influences how much food an organism will require, and also affects how it is able to obtain that food.”

A primary source of energy for cells is glycolysis. This pathway is found in most known organisms. As a glucose molecule is converted to two molecules of pyruvate, free energy is released to form high energy molecules adenosine tri phosphate (ATP) and reduced nicotinamide adenine dinucleotide (NADH). Glycolysis is oxygen independent. Glycolysis is found in most organisms, suggesting this is an ancient pathway. The pyruvate and NADH may be metabolized by atmospheric oxygen in aerobic processes or without oxygen in anaerobic processes.

Glycolysis occurs mostly in cellular cytosol. The Emden-Meyerhof-Parnas EMP pathway describes the most common type of glycolysis. The overall reaction turns each molecule of glucose into two molecules of pyruvate, 2 ATP, and 2 NADH.

In gluconeogenesis, another cellular process, various source molecules are used to generate glucose. These source molecules are typically not carbohydrates. Source molecules can include glucogenic amino acids, lipids such as triglycerides, glycerol, pyruvate and lactate.

Gluconeogenesis is important in maintaining blood glucose levels, avoiding low levels, or hypoglycemia. Other pathways that raise blood glucose levels include glycogenolysis and fatty acid catabolism.

In vertebrates, gluconeogenesis takes place mainly in the liver. In many animals the process occurs during periods of fasting, starvation, low-carbohydrate diets, or intense exercise. Gluconeogenesis is a target of therapy for type 2 diabetes.

From www healthline com/health/diabetes insulin-and-glucagon#Glucosedisorders4 (the link is split here but easy to reconstruct): Glucose is available through a wide variety of foods. During digestion, foods that contain carbohydrates are converted into glucose. Most of this glucose is sent into the bloodstream, causing a rise in blood glucose levels. This increase in blood sugar signals the pancreas to produce insulin. The insulin tells cells throughout your body to take in glucose from the bloodstream. As glucose moves into cells, blood glucose goes down. Some cells use the glucose as energy. Other cells, such as in the liver and muscles, store any excess glucose as glycogen. The body uses glycogen for fuel between meals.

Glucagon is a protein hormone that works to counterbalance the actions of insulin. About 4-6 hours after a meal, the glucose levels in blood decrease, triggering the pancreas to produce glucagon. This signals the liver and muscle cells to change stored glycogen back into glucose. The cells release this glucose into the blood stream, providing a source of energy for cells throughout the body.

In Type 2 diabetes, the body makes insulin but cells don't respond to it normally. They don't take in glucose from the bloodstream, which leads to higher blood sugar levels. Over time, the diabetic body produces less insulin, exacerbating the high blood sugar.

In assessing how a person handles glucose, the glucose tolerance test was first described in 1923 by Jerome Conn. Wikipedia, Glucose Tolerance Test. A common version of this is the Oral Glucose Tolerance Test (OGTT).

Subjects are encouraged to maintain a healthy carbohydrate intake for days before the test. The standard test dose often is not calibrated to body weight, except a person smaller than 43 kg (94 lb) should not be treated as a normal adult. The patient is instructed to fast for 8-12 hours before the test. After a zero time baseline blood level sample, the patient takes a measured dose of a glucose solution, to be consumed within 5 minutes. Blood is drawn at time points for glucose measurement, and sometimes for insulin levels. A typical test samples only sugar, at 0 and 2 hours. Some common protocols test up to 6 hours. The standard challenge drink is 75 grams of glucose. For a 43 kg person (94 lb), this is 1.7 g/kg body weight. For an 86 kg person (188 lb), this is 0.87 g/kg. For a 250 lb person (114 kg), this is 0.66 g/kg.

According to one report, fasting plasma glucose should be below 6.1 mmol/L (110 mg/dl). Fasting levels between 6.1 and 7.0 mmol/L (110 and 125 mg/dL) are borderline (“impaired fasting glycemia”), and fasting levels repeatedly at or above 7.0 mmol/L (126 mg/dL) are diagnostic of diabetes. After the glucose drink and one hour, a glucose level below 10 mmol/L (180 mg/dL) is considered normal. For a 2 hour test, after 75 g intake, a glucose level below 7.8 mmol/L (140 mg/dL) is normal. (End of Healthline citation). Note that 180 mg/dl is 164% and this 140 mg/dl is 128% of the 110 mg/dl expected after fasting.

Glucose tolerance tests are sometimes adjusted to give more glucose for a larger person. A target of about 0.9-1.2 g glucose/kg body weight might be a reasonable range. Glucose tolerance tests are common for pregnant women, often between 24 and 28 weeks of pregnancy. The challenge dose is typically smaller than for a typical OGTT, such as 50 g glucose, or a target of 0.6-0.9 g glucose/kg body weight. For a pregnant woman, fasting levels of 92 mg/dl or greater, and one hour post challenge of 180 mg/dl are normal.

Other perspectives on normal fasting blood sugar cite 72 to 108 mg/dl (4 to 6 mmol/L), or 80 to 120 mg/dl. Some consider 120 mg/dL to be a relevant threshold for high but tolerable normal levels. In short there is a modest variation in what many people would consider normal fasting, and presumably values in this 72 to 120 mg/dl range would likely satisfy most observers as reasonable fasting levels for a normal, healthy individual.

Turning briefly to treatment protocols, in a traditional, older cellular activation protocol, insulin and glucose were added in patterns designed and monitored to maintain blood glucose levels in a target range of 150-200 mg/dl (8.3 to 11 mmol/L) or lower. This range remains interesting in current treatment protocols, as this is a range not unusual after consuming a meal. Comparing this, we see this is near a range typical for an OGTT blood glucose peak (before one hour) and consumption (cf. OGTT 2 hour level of 140 mg/dl, 7.8 mmol/L).

Many types of food are converted by the body to glucose, and to other metabolic components. Complex carbohydrates, such as starches in grains, potatoes, and legumes, are broken down by various pathways. These pathways have various energy requirements, and release glucose in varying time scales. Glucose can be derived from other sources as well. Evolution has found ways to use a wide variety of foods to make a wide variety of metabolic components for the body.

People with Type 2 diabetes sometimes have trouble with insulin correctly inducing cells to take up and process glucose. As individual cells are taking up less glucose, the cumulative blood sugar rises, or when decreasing decreases more slowly. The cells function less well.

### Pulsatile Insulin

For some years, scientists have desired and tested various paths to metabolic stimulation. Administering glucose and insulin in various combinations has been found to stimulate cells that are operating with imperfect insulin response.

Research has demonstrated the natural pattern of secretion of insulin in bursts by pancreatic beta cells in response to a carbohydrate load. A convincing body of evidence indicates that insulin is secreted in synchronized bursts from the pancreas into the hepatic portal vein.
Cook D L. “Isolated islets of Langerhans have slow isolations of electrical activity.” Metabolism. 1983; 32:681-685;
Bergsten P, Grapengiesser E, Gylfe E, Tengholm A, Hellman. “Synchronous oscillations of cytoplasmic Ca²⁺ and insulin release in glucose-stimulated pancreatic islets.” Journal of Biologic Chemistry. 1994; 269:8749-8753;
Zhang M, Goforth P, Bertram R, Sherman A, Satin L. “The Ca²⁺ dynamics of isolated mouse beta-cells and islets: implications for mathematical models.” Biophysical Journal. 2003; 84:2852-2870.

Multiple studies in humans and animals have described the resulting oscillatory nature of systemic levels of blood glucose and insulin.
Lang D A, Matthews D R, Peto J, Turner, R C. “Cyclic oscillations of basal plasma glucose and insulin concentrations in human beings.” The New England Journal of Medicine. 1979; 301:1023-1027.
Matveyenko A V, Veldhuis J D, Butler P C. “Measurement of pulsatile insulin secretion in the rat: direct sampling from the hepatic portal vein.” The American Journal of Physiology. 2008; 295:E569-E574.
Porksen N, Nyholm B, Veldhuis J D, Butler P C, Schmitz O. “In humans at least 75% of insulin secretion arises from punctuated insulin secretory bursts.” The American Journal of Physiology. 1997; 273:E908-E914.
Song S H, McIntyre S S, Shah H, Veldhuis J D, Hayes P C, Butler P C. “Direct measurement of pulsatile insulin from the portal vein in human subjects.” Journal of Clinical Endocrinology and Metabolism. 2000; 85:4491-4499.

Moreover, the loss of pulsatile pancreatic activity, and β-cell dysfunction, may not only be initiating adverse events, but also contribute to the development of hepatic insulin resistance and progression of type 2 diabetes.
Wahren J, Kallas A. “Loss of pulsatile insulin secretion: A factor in the pathogenesis of type 2 diabetes.” Diabetes. 2012; 61:2228-2229.

At least one article notes that individual beta cells in the pancreas release insulin in an oscillatory manner, with a time frame of 2-10 minutes. As an aggregated system, the islets of Langerhans and pancreas each release insulin in the aggregate in an oscillatory fashion, with a cycle time of 3-6 minutes. The article cites changes in calcium concentration as a signal that may be key in synchronizing this. The article notes that after a meal the periodicity of these oscillations remains constant but the amplitude increases.
Hellman B, Gylfe E, Grapengiesser E, Dansk H, Salehi A (2007). “[Insulin oscillations—clinically important rhythm. Antidiabetics should increase the pulsative component of the insulin release]”. Lakartidningen (in Swedish). 104 (32-33): 2236-9, cited in Wikipedia “Insulin Oscillation.”

Providing insulin in a burst fashion using a device delivering small bursts of exogenous insulin would be expected to affect insulin target tissues more effectively by more closely mimicking the secretion of insulin observed in normal individuals. In previous studies, burst intravenous insulin delivery has shown encouraging, yet mixed results in lowering blood glucose levels compared to equal doses of continuously infused insulin.
Matthews D R, Naylor B A, Jones R G. “Pulsatile insulin has greater hypoglycemic effect than continuous delivery.” Diabetes. 1983; 37:617-621.
Komjati M, Bratusch-Marrain P, Waldhausl W. “Superior efficacy of pulsatile versus continuous hormone exposure on hepatic glucose production in vitro.” Endocrinology. 1986; 118:312-319.

Compared to standard therapy, burst or pulse insulin therapy since the late 1980s has shown promising, yet equivocal results in studies examining metabolic control, end-organ damage, and restoration of normal pulsatile pancreatic function in type 2 diabetes.
Aoki T T, Grecu E O, Arcangeli M A, Benbarka M M, Prescott, Jong Ho Ahn J H A. “Chronic intermittent intravenous insulin therapy: a new frontier in diabetes therapy.” Diabetes Technology and Therapeutics. 2001; 3:111-123.
Dailey G E, Boden G H, Creech R H, et al. “Effects of pulsatile intravenous insulin therapy on the progression of diabetic nephropathy.” Metabolism. 2000; 49:1491-1495.
Mirbolooki, M R, Taylor G E, Knutzen V K, Scharp D W, Willcourt R, Lakey J R T. “Pulsatile intravenous insulin therapy: the best practice to reverse diabetic complications?” Medical Hypotheses. 2009; 73:363-369.

Perhaps the most encouraging study on the effects of pulse therapy on end-organ damage showed a significant preservation of renal function by pulsatile insulin infusion.
Weinrauch L A, Sun J, Gleason R E, Boden G H, et al. “Pulsatile intermittent intravenous insulin therapy for attenuation of retinopathy in type 1 diabetes mellitus.” Metabolism Clinical and Experimental. 2010; 59:1429-1434.

These previous studies used earlier variations of what has been called pulsatile intravenous insulin therapy (PIVIT). PIVIT, also referred to as outpatient intravenous insulin therapy (OIVIT), chronic intermittent intravenous insulin infusion (CIIIT), hepatic activation therapy (HAT), metabolic activation therapy (MAT) or the Harvard Protocol, does not replicate the periodicity and amplitude of normal pancreatic function. Most or all of these were insulin centric—add insulin, then add glucose to counter the insulin (titrate glucose to the insulin, driving insulin consumption). A primary feedback parameter is based on RQ—what is the nature of the fuels being consumed by the body?

The so called “Harvard Protocol,” originally was developed at Harvard (Joslin Diabetes Center) by Anton H. Clemens of Miles Laboratory, the inventor of the Miles Biostator® This device was granted FDA clearance K-780393 on Jun. 19, 1978 The protocol for the Biostator® before modification simply infused insulin with a slow peristaltic pump in an amount designed to keep blood glucose in a physiological range, and constantly monitored blood glucose. This required close attention from an operator.

Early Patents—U.S. Pat. No. 4,826,810 (Aoki)

Continuing studies led to a patent by Dr. Thomas Aoki, U.S. Pat. No. 4,826,810, issued May 2, 1989. This is the basis for the “Harvard Protocol.” Claim 1 of this patent is directed to “a human subject having predetermined body tissues characterized by abnormal or impaired dietary fuel processing capability”, which is to say in undesirable mix. In column 6, line 33, the specification notes: “In 8 to 96 hours a significant improvement in the respiratory quotient and carbohydrate oxidation rate should be observed . . . ” comparable to those seen in normals. The import of this seems to be that the treated person starts to look like a normal, healthy person. The patent describes a two phase process, first of a series of ever-rising peaks (increasing baseline), followed some time later by a “secondary series of smaller insulin pulses” to produce a low level oscillation in insulin levels. Treatment continues for one to four days. Col. 5, line 29.

That patent describes a pattern of insulin infusion “producing in the blood supply to said body tissues a series of peaks in the free insulin concentration, said prearranged time periods selected to produce successively increasing free insulin concentration minima between said peaks.” This means that with a burst of administered insulin, the free insulin level rises (Cₘₐₓ), then as the insulin is processed by the body, the level falls back to some degree. The Aoki method administers subsequent insulin infusion relatively quickly to again raise the free insulin concentration in blood, but very importantly for the theory of that invention, the minima of sequential insulin levels successively increase. This means the baseline level of insulin is increasing step by step, with an overlay of still higher pulse levels, both Cₘₐₓ and Cₘᵢₙ. The Aoki patent illustrates in FIG. 1 a series of six peaks, with an increasing baseline highlighted in FIG. 2 of that patent, described as a necessary pattern. When the ramped infusions are stopped, the insulin level declines slowly over additional time. This is a normal body response. The invention further contemplates stimulating oscillation near baseline levels, illustrated in section C in FIG. 1 and detailed in FIG. 3, and continuing this low level stimulation for some time.

This patent cites still earlier work administering insulin based on concurrently measured blood glucose levels. Some of this earlier insulin administration is continuous infusion, sometimes augmented by a larger burst. The invention describes establishing an elevated carbohydrate level, then activating dietary processing with insulin administration in certain patterns.

In this U.S. Pat. No. 4,826,810, an insulin amount was chosen based in an operator's expectations based on subject condition, then glucose was administered in an amount to prevent hypoglycemia. In other words, the amount of glucose to use was chosen to “cover” the added insulin. The intermittent administration of insulin was considered important, and significantly administering insulin in a pattern to continually raise the baseline level of insulin. Insulin bursts were administered in a 10 to 50 mU/kg range (Claims 13, 18, 21) and more particularly in the range of 20-40 mU/kg (Claims 14, 19). This protocol also contemplated a followon insulin administration with bursts of lesser magnitude (Claim 2), after a first insulin burst sequence and a delay sufficient to return insulin levels to a baseline level. Further claims are directed to these followon bursts of insulin to be in the range of 1 to 20 mU/kg (Claims 16, 21). Carbohydrate loading was claimed in the range of 10 to 100 g.

That patent notes wide variation in subject responses. It does note that the insulin pulses are timed to keep the insulin baseline level to keep rising during a first phase of the invention. This phase can last up to three hours, generally within the range of 6 to 180 minutes (180 is 30 times 6—a wide range). The invention notes particularly effective results with a series of 10 pulses, six minutes apart, over an interval of 56 minutes (56/6 is 9.33, so they may have intended 54 minutes after administering a first pulse at 0 minutes and a tenth pulse at 54 minutes. It may be that the 56 minutes contemplated some time for pulse infusion, which would work out to a cumulative 2 minutes, or average 0.2 minutes (12 seconds) per pulse infused.

That patent notes that insulin can be administered by various routes, including IV, IP and subcutaneous, following ingestion of a mixed meal containing 10 to 100 g of “dietary carbohydrate”, or glucose, or infusion of an equivalent combination of metabolic fuels. The specification notes an expected significant improvement 8 to 96 hours later in respiratory quotient and carbohydrate oxidation rate after ingesting a carbohydrate meal. The specification further notes that administering carbohydrates to induce elevated blood glucose levels is contrary to standard treatment regimens. It notes that the specified insulin pulse pattern differed from normal or diabetic responses in common regimens.

In the next Aoki patent using insulin therapy, U.S. Pat. No. 6,579,531, issued Jun. 17, 2003, claim 1 relies on determining a baseline respiratory quotient, treating, and watching for an improvement in RQ. Five more Aoki patents, through U.S. Pat. No. 6,967,191, issued Nov. 22, 2005, also rely on RQ for a primary control parameter.

### Glucose Levels

In historical therapies, such as the Aoki and Harvard protocol development, the amount of carbohydrate was from no added carbohydrate, for example in an already hyperglycemic subject, to some minimal amount determined as needed to cover or offset the administered insulin therapy. Both the carbohydrate and insulin amounts were often modest, and often insufficient to provide a long term therapeutic benefit. This was not intended to be timid or unkind—it was simply cautious, and not understood.

Glucose administration was often “ad lib”—that is “enough glucose to offset insulin injections and keep blood glucose in acceptable ranges.” The relatively low levels of glucose and insulin used in historical methods resulted in a large number of inconsistencies and even failures to achieve more than short term improvement in metabolic status, such as only a day or two. Some few subjects got some benefit, but most “relapsed” fairly soon.

### Pharmacokinetics

The pharmocokinetics of drugs has been closely studied for decades. It is typical that when an active substance is introduced into the bloodstream, various processes will “clear” the active substance. Blood levels of the active substance rise upon administration, and fall upon clearance. The rate of rise and fall depend on many factors such as rate and type of administration, and clearance processes. Administration directly into the bloodstream tends to be the most rapid means of introduction. Clearance processes include consumption (transformation) of the active substance, physical clearance such as filtering out through the kidneys, and degradation such as breakdown in the liver.

The rate of clearance is sometimes characterized by “elimination half life”, which is the time required for the concentration of the active substance to reach one half its original value. If an active substance is given as a bolus or IV push, this will distribute through the body and fairly quickly (minutes) reach “Cₘₐₓ” or maximum concentration. If an active substance is given as an oral dose, or intramuscular injection, or other means, the rate at which the active substance reaches the blood stream is often slower, and Cₘₐₓ is both later and often lower than what is seen with IV administration. Either way, clearance processes start right away to reduce that concentration. Scientists often look at “AUC” (area under the curve, integrating levels over time) to get a better understanding of how much drug is available in the body.

A substance that is rapidly cleared will have a short half life. After some number of half lives, typically 4 or 5, the blood level will drop back to close or very close to baseline, the level before any of the active substance was administered. If a subsequent dose of active substance is administered, the blood level just before that subsequent dose is characterized as Cₘᵢₙ or minimum concentration in this trough in blood level. In the Aoki '810 method, the Cₘᵢₙ levels increase between insulin infusions. Waiting longer for the next infusion allows Cₘᵢₙ to be lower, to increase less or little between infusions.

Insulin is typically consumed rapidly by the cells on which it acts. This is a primary clearance method for insulin. This means that insulin has a short half life for clearance (tens of seconds). Natural insulin is produced in the body, and introduced directly into the blood stream. Diabetics take insulin in various forms, including injectable forms that are designed to reach the blood stream over some moderate period of time, such as tens of minutes.

In general, administering too much insulin can be unhealthy and needs to be controlled by either cutting back on insulin release or by adding glucose, or both. In a healthy person, the pancreas releases a burst of insulin when glucose levels are high, with repeated bursts until the glucose level reaches a level acceptable to the pancreas control systems. As discussed above, glucose levels typically rise significantly after a meal, decreasing over some two to three hours, significantly in response to insulin release into the blood stream.

Glucose is subject to many and complex biological processes in the body. Glucose is consumed by cells for active energy. This is a moderately rapid clearance process. Glucose also is processed in various storage systems. This has a longer half life. The net clearance of glucose is complex, touching many body regulatory systems.

Glucose can be administered directly to the veins, a common procedure in hospitals. Glucose clearance typically has a half life of minutes to tens of minutes.

Glucose is typically derived from eating, or oral dosing. Various processes in the stomach and other body parts lead to a fairly rapid transfer of glucose, first from the most readily available forms, such as glucose in water, which can simply diffuse across various membranes from the gut into the blood stream. Other forms of glucose, such as in sucrose (a disaccharide which the body splits into glucose and fructose) takes a little longer to process, but is a rapid transformation. The fructose is processed by the body to give a second molecule of glucose. As these take a bit of time, the contribution to blood glucose is a bit slower, first to rise initially, then to continue as the sucrose is processed. Complex carbohydrates such as starch are processed still more slowly, and the body has various storage and processing systems. Thus glucose delivery to the blood comes from processes with half lives of seconds to days (reservoirs). There are a great many control systems designed to handle glucose inputs (ingestion) and the body's momentary and long term needs.

### Measuring Effects

Since it is difficult to measure cell operating efficiency directly, scientists have used varying methods to assess cumulative cell effectiveness.

The older outpatient intravenous (IV) insulin therapy (OIVIT) refers to an outpatient regimen that integrates pulsatile or continuous intravenous infusion of insulin via any means, guided by the results of measurement of:
respiratory quotient; and/or
urine urea nitrogen (UUN); and/or
arterial, venous, or capillary glucose; and/or
potassium concentration;
   and performed in scheduled recurring periodic intermittent episodes.

Early work on cellular stimulation by Aoki and others relied on respiratory quotient (RQ) to guide treatment protocols. RQ was important in “the Harvard Protocol”. This also is important in “OIVIT” and other older protocols.

RQ is the ratio of oxygen consumed to carbon dioxide produced in a person. This is a dimensionless number used in calculations of basal metabolic rate (BMR) when estimated from carbon dioxide production. This is a form of indirect calorimetry. RQ is measured using a respirometer. The RQ value indicates which macronutrients are being metabolized, as different energy pathways are used for fats, carbohydrates, and proteins. A value of 0.7 indicates that lipids are being metabolized, 0.8 for proteins, and 1.0 for carbohydrates. The approximate RQ of a mixed diet is 0.8.
Widmaier, Eric P.; Raff, Hershel; Strang, Kevin T. (2016). Vander's Human Phvsiologv: The Mechanisms of Body Function. New York: McGraw Hill.

Other factors that may affect RQ include energy balance, circulating insulin, and insulin sensitivity.
Ellis, Amy C; Hyatt, Tanya C; Gower, Barbara A; Hunter, Gary R (2017-05-02). “Respiratory Quotient Predicts Fat Mass Gain in Premenopausal Women”. Obesity (Silver Spring, Md.). 18 (12): 2255-2259. doi:10.1038/oby.2010.96

For glucose, with the molecular formula, C₆H₁₂O₆, the complete oxidation equation is C₆H₁₂O₆+6 O₂→6 CO₂+6 H₂O. Thus, the RQ=6 CO₂/6 O₂=1.

RQ value corresponds to a caloric value for each liter (L) of CO₂ produced. If O₂ consumption numbers are available, they arc usually used directly, since they are more direct and reliable estimates of energy production. Wikipedia.

This is measured on a machine that measures oxygen and carbon dioxide in a respiratory system. The patient breathes natural or augmented air, and breathes out. The body consumes oxygen and releases carbon dioxide. These gases are not difficult measure. Devices are made to make these measurements. One useful device is a Vacumed Vista-MX2 Metabolic Measurement System.

In the presence of glucose, a normal RQ should be about 0.90 (or higher), or one mole of O₂ consumed per 1.1 moles of CO₂ produced. A normal resting ratio is about 0.85 (=1 O2/1.18 CO₂). A person with impaired glucose processing might have an RQ of 0.75 (=1 O2/1.33 CO₂).

Note that RQ is a ratio, reflecting the type of metabolism, particular what fuels are consumed to provide energy for the body. However this gives no information about the rate or amount of metabolic activity—it is merely a ratio.

The Harvard Protocol, including OIVIT, PIVIT, CIIT, HAT, or MAT, each derivative from or taken in consideration of the Harvard Protocol, all rely on RQ as a primary feedback parameter. As just noted, this does provide information about the type of metabolism. It fails to provide information about the amount of metabolism. This turns out to be a critical oversight, at least in terms of providing a significant and repeatable therapeutic benefit for most people and for longer durations.

The Harvard protocol procedures gave encouraging but mixed results in long term health improvement (persistent benefit several weeks after treatment). Even when using earlier forms of precursors to the present invention, patient outcomes were inconsistent for patients that appeared to be fairly similar. In some patients, the results of burst treatment show improved cellular function. In other patients, the same protocol gives less to little improvement. Prior to this invention, this puzzlement was a continuing challenge to better understand the lack of uniformity with similar stimulation, even as to a single patient.

This variability between patients and variability between treatments given to the same patient was extremely frustrating until the present invention was developed. The present invention gives much more consistent results across a wide variety of patients, and uniform results as to each individual patient. The present invention also gives more consistent and very promising results for a wide variety of conditions.

### SUMMARY OF THE INVENTION

Administering a sizeable quantity of readily metabolizable carbohydrate, accompanied by insulin administered in a pattern more or less along the lines of natural physiological patterns (timing and quantity) has been shown to improve carbohydrate processing in a wide range of individuals, with varying health challenges. This is helpful for diabetics, but also for treating subjects with wounds, mental problems, and much more. Such reports are not unusual, and the goal has been recognized for years. The difference here is that metabolic improvements are longer lasting. Improvement is seen almost all subjects, which is a great improvement over previous treatments that gave inconsistent improvement across subjects and sometimes in the same subject.

A key improvement here is the carbohydrate is given as dextrose (D-glucose). This can pass directly into the blood stream, and is readily metabolized in liver, muscle, and other cells. Another key improvement is the size of the dose. Targeting “a large meal” value of calories, a useful dose is 25% or more of the normal caloric input for a healthy person of that body weight, not considering additional calories for exercise. Preferably the dose is on the high side of this, more than 30% and preferably about 40-55% of that reference level. In addition, the glucose dose is split into sub doses and administered over the duration of the insulin injections. In one preferred example, about 3.5 g dextrose/kg body weight (45% of the daily level) is divided into 9 roughly equal portions, administered as an oral dose portion approximately every 30 minutes. Insulin is provided in microbursts of 10 to 35 mU/kg per burst (and up to double this), every six minutes in a sequence of 10 pulses, then a rest period. Insulin amounts are chosen in anticipation of desirable physiological blood glucose levels, and from measuring blood glucose, adjusting the insulin amounts to target staying in a desirable band of about 150 to 200 mg/dl, typical levels seen in healthy humans about an hour after a typical meal.

This provides a generally level input of dextrose and a generally level input of insulin over each treatment cycle and mostly over the treatment day.

Diabetic patients often do not store hepatic or muscle glycogen as effectively as a non-diabetic. Glycogen provides an energy reservoir that can be converted to blood glucose when blood levels drop. Diabetic patients treated repeatedly with the present invention improve their glycogen storage and release capabilities, in other words in a more normal and healthier manner.

It turns out that a similar treatment for non-diabetic patients improves health for a very wide range of ailments.

The present invention models some important steps on natural human processes, specifically the size and rate of insulin release found in a healthy human. A sizeable dose of glucose is presented, proportional to typical daily intakes. A corresponding amount of insulin is injected in cyclical, pulsatile fashion to provide a continuing stimulus to cell glucose intake by cells during a treatment period. It is preferred to inject insulin in small bursts similar to natural physiology. We often refer to this as “microburst injection.”

A primary thrust of the present invention is to provide a method and mechanism for presenting a sugar substance, such as glucose, and a peptide hormone, such as insulin, in amounts and patterns that have been found to stimulate cellular activity. Among the many benefits of this stimulation are a variety of health benefits. Informally, it is reasonable to think of this method as “jump starting” the cells.

The Microburst Insulin Infusion (MII) of this invention uses insulin to titrate against a delivered amount of glucose. The glucose preferably is adjusted for the patient's body weight with a goal of providing at least a minimum amount of glucose necessary to store glycogen in both liver and muscle. The glucose preferably is administered as D-glucose (dextrose), typically orally. Insulin bursts are sized and sequenced to let insulin levels fall significantly before the next insulin burst. Brief bursts of insulin of 100 or 200 U/mL are preferred, which amounts to typically 2 to 40 microliters injected per burst. Fast acting forms of insulin are preferred. Blood glucose levels are assessed during a treatment cycle.

The present invention introduces several changes over previous methods. These are very effective in combination. We expect that individual components of these changes would be sufficient to improve long term benefit.

First, the carbohydrate load is significantly larger than in previous therapies. The target load is now 100 to 150% of the caloric value of a large meal. Particularly preferred is an amount on the order of 40-50% of the normal daily dietary requirements of a healthy person of the patient's weight. An amount as low as 25% of the daily requirement may be helpful, but generally more than about 33% is preferred.

Second, the carbohydrate is delivered in a closer approximation to continuous—with a typical dose divided into subparts taken during an insulin pulsing series. Preferably this total dose is divided into a significant number of subparts, such as 9 partial doses. In a preferred embodiment, the timing of doses is roughly correlated with the size of the dose. For example, if a first carbohydrate dose is 10% of the total dose for the day, a second dose could be given after about 7 to 15% (approximately 10%) of the time planned for a treatment day. A preferred pattern is 10% at time 0, another 10% at time 30 minutes (12.5% of 4 hours), then 13% at time 60, with or shortly after the last insulin injection of a first treatment cycle. Repeating this three times with a final carbohydrate dose as the last portion of the treatment day gives an approximately linear carbohydrate administration, with an unterminated rest period after the final glucose dose.

Third, preferably this carbohydrate is delivered as dextrose, or D-glucose.

Fourth, there is a higher order time frame in the sequence of repeated treatments. A primary treatment involves the administration of glucose and a series of insulin bursts. In a typical practice, these bursts are administered over about 60 minutes, followed by a rest period. This primary treatment cycle is then repeated, typically three times in a “treatment day.” This primary treatment cycle may be repeated only twice or rarely four times in a treatment day. In preferred practice, a treatment sequence is repeated some days later. Most newly enrolled patients are treated two days in their first week, usually separated by a day or two of rest, and usually two days in the second week, again typically with a day or two of rest between treatment days. Depending on progress, treatment in the third and subsequent weeks will often be one day a week, although for patients significantly out of balance they might be treated twice a week for some time. It is typical for a patient to be treated for twelve sequential weeks.

For framing and terminology, administering glucose and a sequence of insulin bursts in balance with that glucose is called a “treatment” or “treatment cycle.” This is traditionally ten insulin bursts administered over 60 minutes, followed by a rest period of typically 30 minutes. Variations are of course possible and not uncommon. For a typical daily sequence, treatment cycles are repeated usually for a total of three times (can be two to four), as a “treatment day.” Treatment days are repeated according to the patient response, as a “treatment sequence.”

In a typical treatment day, a glucose/insulin treatment cycle, usually repeated three times, provides the caloric equivalent of a large meal over the sequential cycles that day (not per cycle). The patient typically will eat a snack later in the day and a typical meal to round out their eating cycle on a day of treatment.

One particularly useful device for administering controlled dosing of insulin according to the method of this invention is the Bionica Microdose MD-110. This was designed to deliver microbursts of insulin.

Progress in improving patient condition is preferably assessed by measuring the amount of resting carbohydrate metabolism. This is assessed in the course of patient intake evaluation. On a typical day of treatment, this is measured before a treatment cycle, at some point partway through a treatment process, and at the end of a daily treatment cycle grouping. To the extent these amounts are low, a next date for treatment can be fairly soon, even the next day. As amounts improve after multiple days of treatment, treatment days may be two per week, then less often.

Longer term progress can be assessed by evaluating liver condition. In particular, for many patients who start treatment with poor metabolic balance, they have an excess of fat in the liver. This can include people with fatty liver disease. Many people in this condition have poor response to insulin and a poor amount of carbohydrate metabolism. Fatty liver can be observed rather easily by ultrasound. This is a routine test in a radiology clinic. We have observed many patients with a significantly fatty liver to reduce that “visible” fat to decrease significantly. Some 50% of diabetics have significant fatty liver complications. There is no standard treatment that will reliably reduce liver fat. This invention provides a valuable protocol for treating this.

Liver function can also be evaluated by looking at blood sugar levels after a meal. For a patient who is in good balance, we expect and find “typical” blood sugar levels after a typical meal. For an individual monitoring their blood level after a standard or otherwise repeatable meal, if the blood sugar level over time is relatively consistent after such a meal, this is good. If the blood sugar level is inconsistent after various instances of ingesting an equivalent meal, this is sometimes referred to as “brittle”, that is, the body regulation systems are not in balance or working sufficiently well. A positive sign that the inventive method is working well is when an individual is able to get consistent blood sugar responses to an equivalent meal.

### DESCRIPTION OF THE PREFERRED EMBODIMENTS

### Drawings

FIG. 1 is a chart of metabolic flows showing the interaction of glucose, insulin, the liver, the pancreas, and various signals and metabolic flows within this system.

FIG. 2 is chart showing glucose and insulin levels in four experiments, simulated and measured.

FIG. 3 is a chart of measured insulin blood levels after various injections of insulin microbursts.

FIG. 4 is a magnified view of FIG. 3.

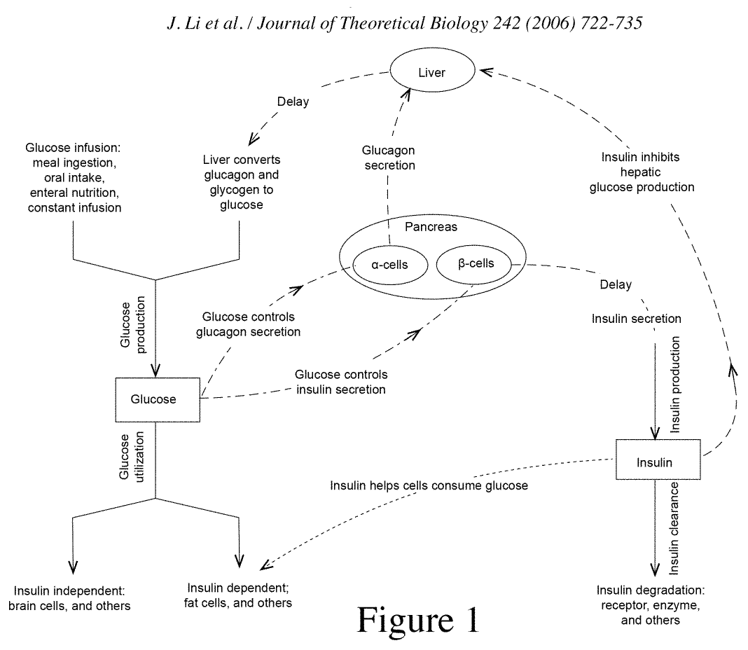

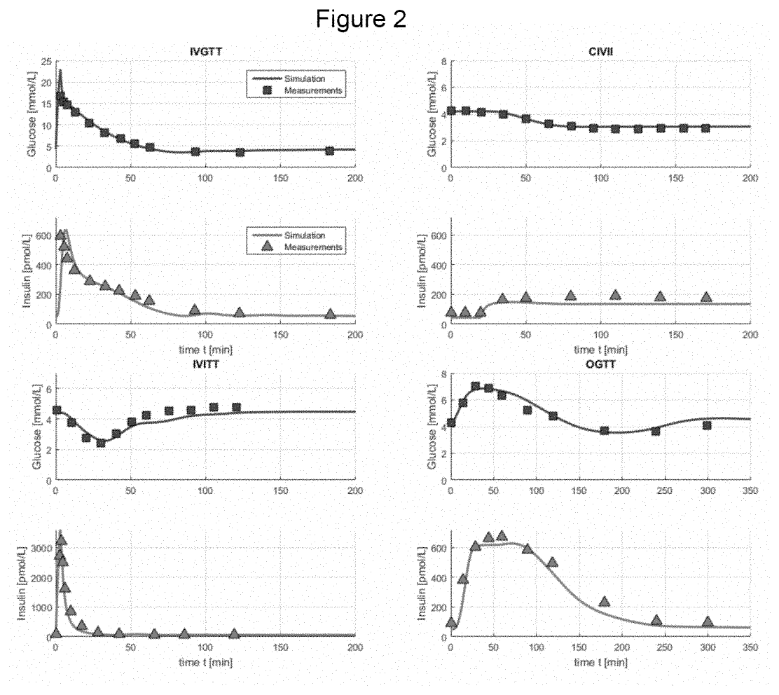

### DETAILED DESCRIPTION OF THE INVENTION

The new theory in this invention is to give carbohydrate nutrition in a large dose with the intention of maximizing liver conversion of glucose to glycogen, but not so much glucose as to drive significant conversion to fat. The insulin pulsing is intended to mimic natural insulin release levels for such a carbohydrate intake. The carbohydrate/insulin administration sequence is designed to bring blood glucose levels above fasting levels and preferably close to what would be expected an hour after a meal. This level is maintained over several hours of a treatment sequence, giving additional carbohydrate and continuing a pattern of insulin bursts.

A useful evaluation tool is an assessment of the amount carbohydrate metabolism (ACM). This is relatively easy to assess from the volume of CO₂ exhaled by the lungs. A respirometer is a common tool for this assessment. This is taken as a resting value, with the patient at rest for a period of time to stabilize this value. The volume of CO₂ exhaled can increase by a factor of 8 with heavy exercise. In a preferred embodiment of the invention, patient carbohydrate metabolism is assessed, at rest, before beginning a treatment day. Typically this is assessed again after a first treatment cycle (after the last insulin burst of that sequence). Typically this is assessed once again after the final treatment cycle of a treatment day.

We expect resting metabolism of carbohydrates to increase during and shortly after a treatment cycle. We expect improved metabolism at the end of a treatment day. The amount of carbohydrate metabolism is used for assessing treatment efficacy, and whether the next treatment day should be relatively sooner or later. Taken together with glucose measurement after the first treatment cycle, this can help with choosing any potential changes in the amount of insulin to infuse for a subsequent treatment cycle.

An improved amount of carbohydrate metabolism (ACM) shows the treatment is having a positive effect. Relatively higher metabolism shows the sequence of treatments is effective, and suggests a subsequent treatment day can be relatively later. Low metabolism and/or poor improvement in a treatment day suggests that a subsequent treatment day should be scheduled fairly soon.

A healthy, male athlete might exhale 0.3 L CO₂ per minute, at rest. An increase to 0.35 L/min after a first treatment cycle would be typical. We anticipate an improvement of about 30% within any given treatment day. This would be on the order of 0.39 L/min for this example.

More generally, it is common to observe exhaled CO2 of 0.22 to 0.32 L/min in a mixed population, before a treatment cycle. For a severely diabetic person, this rate can be 0.12 L/min. It is not unusual for such a patient to improve to 0.15 L/min (125%) over a first treatment day. We usually see such a person raise their rate over a series of treatment days.

### Glucose Timing

One aspect of the present invention is to have the subject consume significant glucose, based on patient body weight, targeted to be equivalent to a large meal or two small meals. This is administered in multiple treatment cycles typically over a period of about four hours. In a preferred form, the daily dose is divided by the number of treatment cycles, typically 3, and further subdivided within each treatment cycle, typically by 3 again, typically into about 9 oral drinks over a treatment day.

The guidance is to give about one large to two small meals worth of calories, typically about 240 to 300 grams dextrose. This represents 960 to 1200 kCal of energy, or in typical dietary parlance, 960 to 1200 calories. This is on the order of 3 to 4 times a typical administration for an oral glucose tolerance test. This is on the order of close to 50% of a typical daily recommended caloric intake, not counting exercise.

For perspective, a fairly common view is that a typical woman should eat about 2000 calories per day, and a typical man about 2500 calories per day plus additional calories as needed for exercise during that time day. To lose one pound of weight per week these should be reduced by about 500 calories per day. This translates to about 30 calories/kg body weight for maintenance, and some 7 calories/kg less for weight reduction. Exercise consumes surprisingly few calories, such as less than 500 calories with heavy exercise. Exercise does typically improve body functioning and metabolism. Resting metabolism is usually not changed significantly by exercise in the short term. Resting metabolism does improve over days and weeks of repeated exercise.

Targeting “a large meal” value of calories, a useful dose is 25% or more of the normal caloric input for a healthy person of that body weight, not considering additional calories for exercise. Preferably the dose is on the high side of this, more than 30% and preferably about 40-55% of that reference level. In addition, the glucose dose is split into sub doses and administered over the duration of the insulin injections. In one preferred example, about 3.5 g dextrose/kg body weight (45% of the daily level) is divided into 9 roughly equal portions, administered as an oral dose portion approximately every 30 minutes.

In one preferred pattern, each treatment dose is administered as 30-30-40, which is to say 30% of the dose for that treatment cycle, followed by another 30% at about 30 minutes, and a final 40% at about 60 minutes, or about the time of or after the last insulin burst of that sequence. For a daily dose of 300 g, divided by 3, this works out to 30, 30, 40 grams (for 100 grams in the treatment cycle), then repeated for two more cycles. See Table 1 for one example of such a sequence. A dose is given at a certain time. The subsequent dose is given after a time period (here 30 minutes each).

TABLE 1
Example: (dose at beginning of time period)

| Hours | | Dose (g) | | |
|---|---|---|---|---|
| (take next | | (beginning of | | |
| dose) | % of time | time period) | % of dose | % dose/% time |
| 0.5 | 12.2 | 30 | 10 | 0.82 |
| 0.5 | 24.4 | 30 | 20 | 0.82 |
| 0.5 | 36.6 | 40 | 33.3 | 0.91 |
| 0.5 | 48.8 | 30 | 43.3 | 0.89 |
| 0.5 | 61.0 | 30 | 53.3 | 0.87 |
| 0.5 | 73.2 | 40 | 66.7 | 0.91 |
| 0.5 | 85.4 | 30 | 76.7 | 0.90 |
| 0.5 | 97.6 | 30 | 86.7 | 0.89 |
| 0.1 | 100 | 40 | 100 | 1 |
| 4.1 | Total | 300 | Total | |

This could be divided even further, although an initial glucose loading dose of some 5-10% of the planned amount for the treatment day is generally preferred. One preferred embodiment looks to have some 10% of the glucose on board within the first 15 minutes or so of treatment, then most any pattern that will keep the glucose administration more or less ahead of the insulin administration. Table 1 details one such scenario. One possible pattern would be 15 grams (5%) at time zero, then starting the insulin administration, then another 15 grams glucose by about 12 minutes of starting the insulin, then continuing at about 1 g per minute over the next 70 minutes (15+15+70=100 g=33% of day), with the 1 g/minute divided into convenient units. This might be 6 grams every 6 minutes, which might be timed to be 3 minutes before each insulin burst, as just one example. Starting with 5 grams (1.7%) at time zero, followed by 1.5 gram per minute for 63.3 minutes would be another reasonable protocol.

The pattern of glucose administration can vary widely. Giving all of the glucose at one time (it takes several minutes to consume this drink) would be equivalent to a single meal. Giving the glucose in smaller doses, spread out in time, is similar to a patient taking smaller meals, but still adding up to the desired intake. Recent work suggests it is helpful to further divide the glucose dosing into moderately small units, taken with corresponding higher frequency. Dividing 300 grams of glucose into 9 equal portions gives 33.3 grams per dose. Using a 33.3% solution, 33.3 grams would be in 100 ml of drink, or 3.4 fluid ounces, which is 0.38 cup. Spread evenly over four hours, this would be 3.4 fl. oz. every 24 minutes. The total quantity would be 30 fl. oz, or 0.95 quarts (900 ml), over 4 hours. The point here is to spread the glucose into somewhat smaller units, approaching continuous. This resembles continuous snacking, in small doses. The specific pattern is not particularly important. The dosing units do not need to be identical, and the timing can be varied.

Taken at the margin, this suggests considering administering glucose IV. Giving 300 g glucose by vein requires a large volume. Even if using 50% glucose, this would be 600 mL (2.5 mL/min for 4 hours). Our experience is that IV glucose raises levels more quickly than oral ingestion. This can also mean that the oral glucose is not 100% bioavailable. We typically reduce the administered glucose by about 33% when given by IV. IV administration can provide an alternate for any portion of the typical oral dosing (scaled as noted), from 0 to 100% of the glucose dose. This can be given as a continuous infusion, or according to any convenient pattern, noting the guidance to keep blood glucose levels in the tolerable range for this treatment.

The present invention delivers the glucose preferably in the form of D-glucose, often called dextrose. This is easily turned into hepatic glycogen to become a ready reservoir for modulating hypoglycemia (low blood sugar). Dextrose is readily metabolized and can be used as a primary energy source directly in cells of the body. From a typical meal in ordinary eating, some portion of the intake is converted to glycogen. Providing dextrose makes this glycogen production more direct, utilizing a primary metabolic pathway.

The classic OGTT asks patients to drink a solution of fairly concentrated glucose in a relatively short time. The purpose of the test is to give a bolus of glucose to the system. The classic 75 g glucose, if in a 33% solution, would require ingesting 225 ml of solution. This is 0.95 cup, 7.6 fluid ounces. They are asked to drink this within five minutes, more or less as quickly as convenient and tolerable. Our old technique was to have people consume a drink of suitable size, which in many instances was even larger than this. Particularly in our old technique of using a single carbohydrate meal for each insulin injection sequence, this meant ingesting that carbohydrate at the same time (in a period of few minutes, as fast as the patient could conveniently drink it). For many people, consuming a large quantity of GLUCOLA (or our newer dextrose drink) is not well tolerated, causing some nausea. This can be offset by antinausea drugs, easily injected IV since the insulin feed is already in an active vein. The new inventive protocol of subdividing the glucose dose minimizes the nausea complications, among other benefits.

### Insulin Pattern

Medical science has known for decades that insulin is released in the normal body in a cyclical pattern with a time frame of minutes, typically 3 to 6 or 7 minutes. Pulses are released by the pancreas into the hepatic vein, which is directly into the liver. Much of this insulin is used directly by the liver.

Insulin in this invention preferably is provided in an amount to “cover” the glucose (to manage blood sugar levels to tolerable and preferably to typical levels) and encourage proper carbohydrate metabolism. The protocol insulin administration pattern is structured to induce body cells to take up the amount of glucose given. Since the glucose is provided in readily available form (soluble, rapidly absorbed into the blood stream, raising blood glucose levels, with glucose readily available to cells throughout the body), the insulin bursts induce cells in the body to internalize glucose. We are particularly interested in enabling and encouraging liver cells to process glucose. Glucose is provided in generous amounts. This encourages cells to process the glucose at a brisk pace and over several hours, thereby stimulating cellular metabolism generally.

In a preferred embodiment of the invention, the injected insulin is fast acting. Particularly useful products include Humalog (insulin lispro) and Novolog (insulin aspart). Other types of insulin not designed for slow uptake or sustained release may be useful. This follows from seeking to reproduce natural body functions. In current treatment of diabetes, it is not uncommon for diabetics to use a slow release insulin to maintain general blood levels. It is not uncommon for diabetics to use a fast acting insulin such as Humalog together with a meal to provide additional insulin in balance with an expected meal intake.

In the preferred embodiment, it is preferred to start with giving some glucose before starting the insulin administration and to keep the glucose consumption mostly ahead of the insulin administration rate. Starting glucose and insulin more or less together, at about the same time, is fine. In one preferred practice, we start a patient with 10% of the planned daily dose of glucose, starting the insulin bursts at about the same time. The patient gets another 10% glucose after 30 minutes and another 13% after 60 minutes. The insulin plan is divided by 30 (three cycles of 10 bursts), so 5 bursts (17%) are given during the first 30 minutes (against 10% of the glucose, but this is quickly supplemented to 20% at 30 minutes). After the first hour, 33% of the insulin plan has been administered, and the third glucose administration brings the glucose load to the same 33%. This pattern continues in remaining treatment cycles.

In this example, insulin is “ahead” of the glucose from about 18 minutes (after fourth pulse, insulin proportion to this time out of the total insulin and time for the day is higher than added glucose) to 30 and again from 48 to 60, then the next glucose portion puts the glucose ahead. Note that insulin blood levels change quite quickly (seconds to tens of seconds) while glucose levels change much more slowly (tens of minutes), so the subject will not be hypoglycemic unless the insulin amount generally is set too high. This is part of the reason we start with a low insulin burst size, as the body can tolerate moderately high blood sugar for some hours without significant complication.

The amount of insulin is titrated against observed glucose consumption. The initial insulin level is chosen to anticipate using some but not all of the ingested glucose. Blood glucose is evaluated after 60 minutes. In a preferred embodiment, if it is in the 150-200 mg/dl range typical an hour after a glucose tolerance test, the selected insulin infusion level is reasonable. If it is above 200, insulin administration should be increased. If blood glucose is close to 200, some increase in insulin quantity can be considered. If it is below 150, insulin can be reduced or slowed. If it is below 100, insulin can be stopped. If it is below 70 mg/dl insulin should be stopped quickly, and consider adding some dextrose IV.

Natural insulin release from the pancreas has been observed in bursts spaced by about 4 to 6 minutes. In our typical practice, we have settled on spacing of 6 minutes and a sequence of 10 bursts over one hour (burst 1 at time 0, burst 10 at time 54 minutes, with the typical post-burst 6 minutes rounding out 60 minutes). There is considerable room for variation around this. We have used burst spacing of 4 to 7 minutes, and an even wider range would not preclude operation of the invention. Bursts can be spaced more closely. Closer spacing will increase insulin levels during this closer spacing (for the same size injection), which for example can be useful to counteract hyperglycemia. Burst spacing does not have to be regular. Burst amounts do not have to be identical. Bursts do not need to be limited to 10 in a row or to 60 minutes for a treatment cycle. More discussion of burst timing can be seen in U.S. patent application Ser. No. 13/999,463, in connection with FIG. 10 there.

We have tested sequences of 15 bursts, at 6 minute intervals, followed by rest and a second such sequence of 15 bursts. This worked essentially as well as the usual three cycles of 10 pulses each. In earlier work, carbohydrate was given as a single dose. A burst sequence that cumulatively delivers more insulin, such as an extended or longer burst sequence, calls for a larger carbohydrate dose. Our old method of one drink per injection cycle meant some of these doses would have to be rather large. The new inventive protocol of subdividing the glucose dose minimizes the frequently seen nausea complications following a large dose of glucose drink, among other benefits.

Burst spacing of 5 minutes works well, for a wide range in the number of bursts. This also is conveniently given as a sequence of 10 bursts, or could be in some additional bursts, averaging somewhat lower amplitude across the series. In general, we have found the standard burst sequence of ten bursts spaced at 6 minutes to be easy to administer and to give good results.

As noted in the summary, one particularly useful device for administering controlled dosing of insulin according to the method of this invention is the Bionica Microdose MD-110. This was designed to deliver microbursts of insulin. A “micro” burst is a small, controlled injection. The MD-110 can accurately and repeatably inject 10 microliters (2 U insulin when using 200 U/mL) to 200 microliters or more. This injection device is more thoroughly described in U.S. patent application Ser. No. 13/999,463, published Aug. 28, 2014 as US 2014/0243261. This application, by the current inventor, is incorporated herein in full by reference.

Other insulin management systems could be useful with the glucose dosing system. Medtronic has announced an “artificial pancreas system”. This includes the MiniMed 670G, which tracks a patient's blood glucose and releases insulin every 5 minutes based on real-time data gathered from the sensor. Fierce Biotech, Jun. 7, 2017, fiercebiotech-com/medtech/medtronic-s-long-awaited-artificial-pancreas-makes-u-s-debut. We don't know what is the algorithm or regulation guidance of this device.

We would expect a suitable device to be programmable to introduce insulin to manage blood glucose in a selected range of blood glucose. If that band is set to 150-200 mg/dl and the patient is fed on only glucose (dextrose) at the moderately high caloric load of this invention, extended over several hours in line with the practice of this invention, and insulin introduction is relatively rapid and pulsatile, this also should provide benefit to patients over time. Such programming may or may not be readily available to the user or the manufacturer. The advertising materials suggest that the system can be set to work around a configurable set point. This might be set to, for example, some value between 150 and 200, such as 160 to 175.

We know that directly injecting fast acting insulin in short, significant bursts is effective in practicing the present invention. If the insulin pulsing from an alternative pump is already brief and significant, this may require minimal adjustment. If the insulin pulsing is smaller than as taught here, or choice of insulin injected is slow acting (which would smooth out insulin blood levels, which is not the preference of this invention), the alternative device might benefit from more extensive reconfiguration or reprogramming.

### More Detail

A typical sugar dosing is 3.3 to 3.7 g dextrose per kg patient body weight, or 3.5±0.2 (6%). At 4 kCal/g glucose, this is 14±1 kCal/kg body weight. Chemistry kCal (kilo calories) are referred to simply as calories in typical dietary parlance. A wider range of dosing can be useful and effective. It remains important to provide adequate added insulin to balance the amount of glucose dosing.

Using 300 g dextrose for a typical 200 pound (91 kg) person works out to 3.3 g (13.2 kCal) per kg body weight, or 1.5 g (6 kCal) per pound body weight. In a preferred embodiment, this can vary significantly and still be effective. It is not easy to state a minimal effective level, as some individuals will benefit from a modest amount. It is not easy to state a maximum tolerable amount. These are being further explored.

Using 4 g dextrose/kg body weight is reasonable (115% of nominal for the present invention), and even 7 g/kg (200% of nominal) can be used, always with a suitable insulin administration. Modestly less than 3.5 g/kg, such as 3 g/kg (86% of nominal), is likely to be useful at least in some patients. In contrast, using 1 g/kg (28% of nominal) will give some effect, but is not a strong stimulus to the patient. This has been shown over the last 30 years to provide inconsistent results as patient outcomes are sometimes promising but generally not uniform, with significantly variable long term maintenance of benefit, and many patients who show little or no benefit at all.

If the amount of carbohydrate metabolism shows poor carbohydrate utilization, the glucose challenge should be increased, with a corresponding increase in insulin. It is not outside the scope of this invention to allow high blood glucose which is often taken to indicate hyperglycemia. The metabolic stimulation by glucose in the form of dextrose on top of an already high blood glucose is counterintuitive but nevertheless is shown beneficial for therapeutic effect. Accordingly, unlike prior approaches, dextrose is administered even when starting from hyperglycemia because this added dextrose provides one of the necessary signals to the liver and tissues to induce increased carbohydrate metabolism.

This glucose dosing is divided over a daily treatment cycle. As a typical daily cycle includes three glucose/insulin/rest cycles, this means the nominal dose is on the order of 1.1 g glucose/kg body weight per treatment cycle. For our nominal 200 pound person (typical male), this is 100 g dextrose per treatment cycle, some 33% more than a typical glucose tolerance test (OGTT) amount of 75 g. Note however that the OGTT is administered with a single such glucose test, then without additional insulin watching blood levels for up to six hours. In MII three glucose doses are administered within about 4 hours—considerably more than in OGTT. notably offset by infusion of extra insulin.

For a 140 pound person (not unusual for a woman, or a smaller man), the MII dose of 80 g per administration (240 g total, 3.7 g/kg for the day) is only a bit more than the typical OGTT amount. At 3.5 g glucose/kg body weight, this 140 pound person gets 76 g glucose per cycle, or 230 g total for the day. For a 100 pound person (45 kg) at 3.5 g glucose/kg, this is 53 g per dose, 160 g total for the day. A variation in carbohydrate dosing of 5% is not significant in calculating how to dose an individual, and a variation of 10% is likely generally acceptable. For example, for this 140 pound person, the 3.7 g dextrose/kg target value in the range 3.5-3.7 (±5%) should give acceptable results, and in the range 3.3-3.9 (±10%) is likely to be effective. Insulin levels still need to be adjusted per guidance of this invention.

The insulin is given on a fixed pattern, in a preferred embodiment. In general, the protocol is designed to let blood insulin levels fall significantly between insulin bursts, again in a preferred embodiment. This is targeted to give an insulin dose of a selected size, as a rapid push (seconds), and repeated at a repeat time, typically six minutes. In variations on the invention, included in the scope of the invention, this insulin dosing can be varied significantly, in peak height, spacing between peaks, inconstant spacing between peaks, blood level between peaks (Cₘᵢₙ), regular vs. irregular patterns, number of bursts, and duration of treatment.

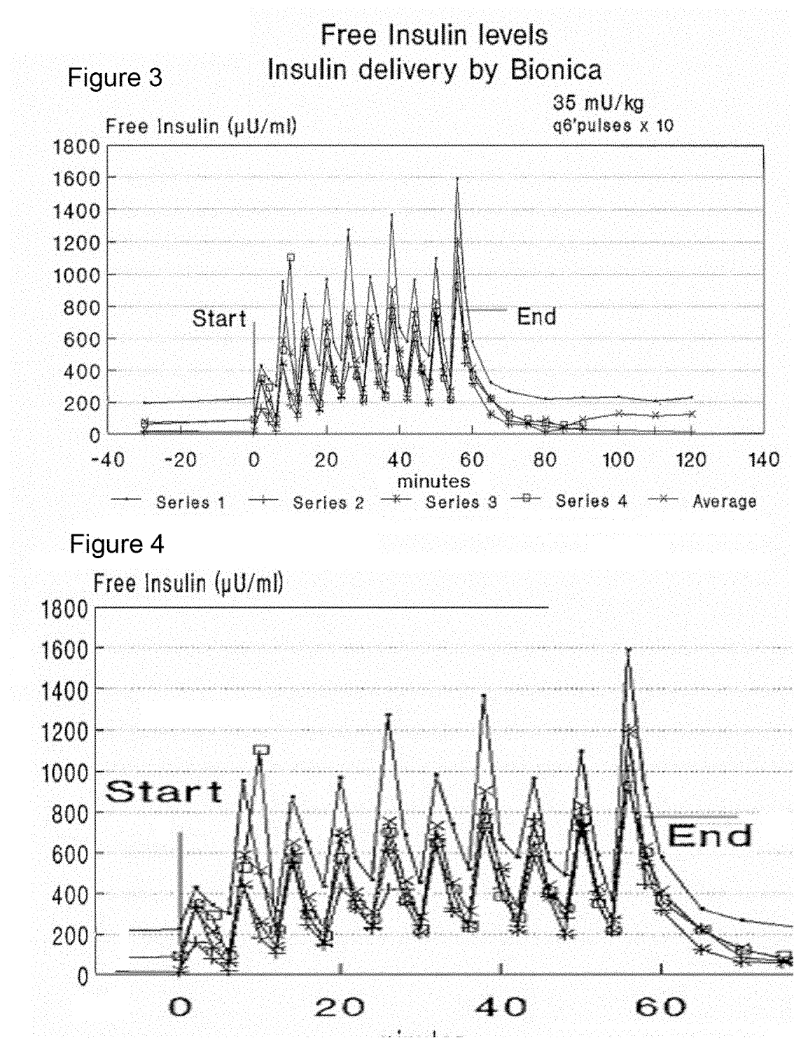

As an actual example, note FIG. 3, which shows blood insulin levels on pulsing at 35 mU/kg every six minutes for ten bursts. Note that 35 mU/kg is on the high side of what is usually given as a patient has undergone several treatment days. As expected from pharmacokinetics, the first burst is distributed through the blood volume, reaching Cₘₐₓ at about 2 minutes, then starts decreasing rapidly to Cmin before a next burst. A second burst before the insulin concentration has dropped to zero means the next insulin infusion adds to the residual insulin, reaching a second Cₘₐₓ somewhat higher than the first Cₘₐₓ. This falls away to a Cₘᵢₙ, which also is somewhat higher than the previous Cₘᵢₙ. This incremental increase continues until by the fourth burst in this example, a steady state is reached. After each subsequent burst, the Cₘₐₓ. is relatively consistently in a narrow band, and the Cₘᵢₙ before each subsequent burst also is in a generally narrow band. This Cₘᵢₙ band is somewhat higher than the resting or zero insulin state.

Remember that this insulin infusion is balanced against a glucose load which is significantly elevated from resting levels. The asymptotic Cₘᵢₙ level reflects a balance among many variables, including the insulin pulsing and the glucose loading patterns. This is expected to change as various inputs are changed. Less glucose loading is likely to raise the asymptotic Cₘᵢₙ (and Cₘₐₓ) as there is less glucose to drive into cells.

In the preferred embodiment 30-30-40 pattern of glucose dosing, the 40% (of that treatment cycle) at time 60 minutes, typically about the tie of the last insulin burst, is intended to clear the cumulative insulin levels by adding a modest excess of glucose.

Insulin levels will be different in a healthy or a diabetic patient.

In traditional PK (pharmacokinetic) analysis, we would expect giving a lower burst amount would give a lower Cₘₐₓ and a lower Cₘᵢₙ before a subsequent burst. Saturation/steady state levels tend to be related to general physiological conditions, so we would expect to see the same asymptotic Cₘᵢₙ if this is a system which saturates. This would take more bursts to reach when using a smaller input. This might be balanced or offset by clearance mechanisms that would cause this asymptotic Cₘᵢₙ to be somewhat different when giving a smaller burst amount. Similarly, were a larger burst amount (or frequency) to be used, we would expect the Cₘᵢₙ steps to increase faster. The asymptotic Cₘᵢₙ that may be the same as observed with 35 mU/kg, or perhaps somewhat different.

It is preferable for this invention to keep blood glucose mostly within a desired range, preferably approximately a typical range for blood glucose some time after a meal. Blood glucose levels of 150 to 200 mg/dl are favored. Some range outside this is still acceptable.

Hypoglycemia is undesirable, and easily life threatening. Blood glucose levels below about 70-80 g/dl are generally considered low. In the preferred method, levels below 100 g/dl during a treatment day are considered low. If such low levels are observed during treatment, the rate of glucose administration should be increased, or the rate of insulin administration should be reduced, or both. Similarly hyperglycemia is undesirable, but can be tolerated for a longer time. Particularly when treating a diabetic patient with generally high blood sugar levels, blood glucose above 200 mg/dl, even significantly above 200, may be seen. According to the teachings of this invention, it is still appropriate to administer significant additional glucose, but the insulin bursts will be adjusted to attempt to bring the blood glucose levels below 200, at least during the course of a treatment day.

A cycle of dextrose administration and insulin pulsing (at a dose size and repeat time) typically takes about 60 minutes. The patient is allowed to rest, then undergoes a second treatment cycle. For a typical patient, this is followed by a third treatment cycle. Treating for 60 minutes with a 30 minute rest period, then 60-30-60 minutes is a total of about 4 hours.

Depending on a treatment protocol, this cycle may be repeated the next day, or in some small number of days, or up to about 30 days before the next cycle. The more frequent daily repeats are for patients that are more out of balance, or in other words not as healthy. A healthy person will show a consistent and expected response to glucose and insulin dosing. These patients can repeat the protocol after 14-28 days. If the protocol results over some few treatments are in the expected normal range, the treatment can be considered sufficient.

For insulin amounts, the target per dose is 10 to 35 mU/kg, with a nominal maximum of about 70 mU/kg in preferred practice. For this range of 10 to 70 mU per burst, at 10 bursts per treatment cycle this is 100 to 700 mU, and over three treatment cycles, this is 300 to 2100 mU, per kilogram per patient per day. For a 200 pound (91 kg) person, 35 mU/kg is 3185 mU or 3.2 units per burst. Over 10 cycles in a treatment cycle, this is 32 units, and over three treatment cycles per treatment day this is 96 units. This amount is some three times what a moderate sized, healthy person releases in a normal day, and for about twice the caloric input (normal 32 units for 2000 calories, treatment 96 units for about 1000 calories). For a large person (our 200 pound person), this is a not unusual amount of insulin for a diabetic person to produce naturally, per day. So this is within the general range of insulin release for some people. Looking instead at 10 mU/kg/burst, the total for the day is about 27 U, which is close to that daily 32 U release for a healthy, moderate sized person.

The Bionica Microdose MD-10 is well suited to deliver insulin in microbursts. To deliver 10 mU/kg for a 63 kg person, this is 0.63 U/burst. Giving commercial insulin at 100 U/mL, this calls for 6.3 uL per burst. When treating a 63 kg person at 35 mU/kg, a burst would be 22 uL. At our usual maximum, extended dose of 70 mU/kg, a burst would be 44 ul. For a larger person, such as 126 kg (280 lbs), where it is not unusual to find diabetic conditions or other health concerns, a burst at 70 mU/kg would be 88 ul. The MD-110 typically gives injected bursts in 1 to 10 seconds. Repeatability and accuracy is high. This translates to an injection rate on the order of 5-10 uL/second in the pulses. Commercial insulin also is available at 200 mU/mL. This more concentrated form may be more useful when treating a patient needing higher insulin levels, and particularly for a larger patient.

In a preferred embodiment, patients are usually started at 10 mU/kg. If insulin is being well taken up in the body, this should let the insulin level drop to baseline before the next bolus in the typical sequence of this invention. Testing blood glucose levels after a treatment cycle will show if the glucose is being consumed sufficiently. To induce more glucose consumption, the insulin administered can be increased. The most significant test is not glucose levels but rather the amount of carbohydrate metabolism.

In a normal person, insulin blood levels vary between about 5 to 40 mU/L. Blood levels are often <25 mU/L. Thirty minutes after glucose administration (OGTT), a level of up to 230 mU/L is not surprising, after 1 hour up to 276 mU/L and after 2 hours up to 166 mU/L, and after 3 hours back to baseline. In one report, insulin is measured over a wide range, including levels in some patients that are roughly the same as baseline 16 to 30 mU/L. At a typical blood volume of 5 L, 25 mU/L is 125 mU in the bloodstream, and 230 mU/L is 1150 mU. For a typical person with interstitial fluid of about twice the blood volume (an additional 10 L), at 15 L, 25 mU/L is 375 mU and 230 mU/L is 3.4 U. This 3.4 units is on the order of 10% of what is secreted by a lean, healthy individual during a day. This is consistent with insulin having a short half life. Clearance is likely dominated by consumption.

From another perspective, average secretion of 30 to 50 units from the pancreas per day is not unusual. Insulin reserves within the pancreas can be 350 units. A lean healthy individual might secrete 35 units a day. An obese, insulin-resistant person might need to produce and secrete more than 100 units daily.

A lean male might weigh about 140 pounds, or 63 kg. A typical recommended diet of 2000 calories per day corresponds to 500 g glucose, if the daily carbohydrates were taken only as glucose. With a typical daily release of 30 U of insulin, this is 60 mU insulin/g “glucose”.

According to the method of this invention, a reasonable carbohydrate dose for this person under the treatment regimen is about 225 g, which is 900 calories. This correlates to about 45% of a typical recommended 2000 calories per day. Adding insulin bursts at 10 mU/kg, this adds up to 19 U against the 225 g, for a ratio of 90 mU insulin/g glucose. This is somewhat higher than insulin produced naturally, but only by some 50%. If such a person was less healthy and required more insulin to promote glucose clearance by uptake, infusing insulin at 35 mU/kg/burst, for the day this would add up to 67 U insulin, and an insulin/glucose ratio of 300, almost ten times what a normal healthy person would need, but still less than a significantly diabetic person would require.

So we see that the insulin administration of about 10 to about 50 mU/kg/burst against this added glucose is on the order of the rates seen in typical patients with a range of health conditions. For healthy people, lower levels such as 10-15 mU/kg/burst should be enough. For diabetic people, levels such as 35-50 mU/kg/burst are not surprising. For a deeply out of balance physiology, levels such as 70 mU/kg/burst are sometimes indicated. If the patient responds as many do to the regimen of this invention, their body starts to adjust after a few treatment days and the insulin required to achieve acceptable blood glucose levels will get closer to 50 mU/kg/burst and over time still lower.

The repeat time between insulin bursts can be adjusted so that the individual patient utilizes each insulin burst essentially completely. Although insulin blood levels can be tested directly, this normally requires special equipment, time, and laboratory costs. From experience, if the expected results are achieved, this is well correlated with insulin levels returning to baseline (more or less) between insulin bursts and certainly during rest periods.

### EXAMPLES

All studies used MII which consisted of 3 one-hour insulin treatments, during which infused, controlled microbursts of IV insulin via the Bionica Microdose Pump, are given to match a pre-set measured oral ingestion of glucose equaling the average caloric need for that patient's weight during the period of treatment. A rest period of up to 30 minutes is given between the one-hour microburst infusions. The treatment was calculated and delivered according to the method of this invention. Patients in all three studies continued to receive their regular regimen of hypoglycemic medication either oral or insulin.

Restoration of more normal resting metabolism of carbohydrates was assessed using the Vacumed Vista-MX2 Metabolic Measurement System, a breath-by-breath mixing chamber measurement of the volume of carbon dioxide produced at rest. The volume of CO₂ exhaled corresponds to the amount of carbohydrate metabolism or utilization. The resting carbohydrate utilization measurements were taken 3 times per treatment day. The first measurement is before initiation of MII treatment, then partway through the treatment and then at the finish of the treatment day, after the last treatment cycle. Changes in VCO₂ are diagnostic, and are used to verify patient resting metabolism responses to the inventive protocol, and to determine the necessary treatment frequency (from twice a week to once per week to once per three weeks or longer). A significant increase in VCO₂ after the first hour of treatment indicates that the number of days between treatment may be extended from the current number. By contrast, a small increase in VCO₂ indicates the treatment effect is low and the next treatment should be relatively soon.

The individual treatment protocol for each patient continues until the patient's metabolic status is maintained over a personalized period of time as shown by the ability to quickly increase carbohydrate utilization in the presence of an oral glycemic load during the first hour of treatment. The first 2 treatments are given within 1-3 days of each other. After this, treatment is not continuous or pre-set as to the number of days between treatments.

### Carbohydrate Metabolism Study

Indirect calorimetry is a primary method for metabolic rate measurement. This commonly involves measurement of oxygen consumption (VO₂) and carbon dioxide production (VCO₂).
Haugen H A, Chan L N, Li F. Indirect Calorimetry: A Practical Guide for Clinicians. Nutrition in Clinical Practice. 2007; 22377-388.
   The relation between these gases and metabolic rate is defined by the respiratory exchange ratio (RER), Resting Metabolic Rate (RMR), and other measures. RER, which is a measurement of carbon dioxide produced per unit of oxygen (VCO₂/VO₂), varies with type of metabolic, food substrate (carbohydrate, fat, protein). An increased RER value is indicative of carbohydrate oxidation being favored over the oxidation of lipids for energy production. RMR is the measurement of energy required to maintain basic body functions while in a state of rest. This accounts for 65% to 75% of total energy expenditure.

The aim of this study was to examine the effects of MII therapy on metabolic rate measures as determined by indirect calorimetry. MII treatment significantly increased oxygen consumption and energy expenditure by promoting a greater carbohydrate oxidation rate both in absolute (CHO oxidation in kcals per liter of O2 consumed and CHO oxidation in kcals per minute) and relative terms (percentage of CHO kcals expended relative to the treatment groups total kcals expended per minute at rest).

The increase in the level of carbohydrate metabolism soon after the introduction of microburst insulin therapy is generally observed. Microburst insulin promotes glucose uptake and carbohydrate oxidation while reducing lipolysis and lipid oxidation. These effects are reduced by insulin resistance, yet are still present in patients with type 2 diabetes.
Laville M, Rigalleau V, Beylot, M. Respective role of plasma nonesterified fatty acid oxidation and total lipid oxidation in lipid-induced insulin resistance. Metabolism. 1995; 445:639-644.
Taskinen M R, Bogardus C, Kennedy A, Howard BV. Multiple disturbances of free fatty acid metabolism in noninsulin-dependent diabetes. Effect of oral hypoglycaemic therapy. Journal of Clinical Investigation. 1985; 762:637-644.

A raised RER shows that insulin is favoring carbohydrate oxidation rather than lipids for energy production.

Previous investigators have reported decreases in resting metabolic rate over the short-term (2-8 days) in patients treated with insulin in other treatment regimens not practicing this invention. It is thought this is chiefly due to the lack of stored glycogen and thus the inability to help regulate energy within the body. This decrease is effectively a failure of a treatment to sustain improved carbohydrate metabolism and shows why a failure to induce sufficiently therapeutically improved outcomes is logical.
Buscemia S, Donatelli M, Grosso G, Vasto S, et al. Resting energy expenditure in type 2 diabetic patients and the effect of insulin bolus. Diabetes Research and Clinical Practice. 2014; 106:605-610.
Gonzales C, Fagour C, Maury E, Cherifi B, et al. Early changes in respiratory quotient and resting energy expenditure predict later weight changes in patients treated for poorly controlled type 2 diabetes. Diabetes & Metabolism. 2014; 40:299-304.
Fagour C, Gonzalez C, Suberville C, Higueret P, et al. Early decrease in resting energy expenditure with bedtime insulin therapy. Diabetes & Metabolism. 2009; 35:332-335.

It is thought that the immediate decreases in RMR may be due to the short-term underlying effects of insulin on the reduction of energy-consuming processes such as proteolysis and gluconeogenesis.
Nair K S, Garrow J S, Ford C, Mahler R F, Halliday D. Effect of poor diabetic control and obesity on whole body protein metabolism in man. Diabetologia. 1983; 255:400-403.

We also observe a quick decrease in RMR in the second period following MII, consistent with previous findings. However, there was a subsequent increase in the last period, and a very significant increase of 29% in RMR values baseline-to-baseline over some months and multiple treatment days. This shows more lasting effects of MII compared to prior treatments.

These results suggest that MII has a dramatic relatively long term (in excess of 7-21 days) effect on carbohydrate metabolism, overcoming the reduction of resting metabolic rate seen with conventional insulin treatments. This is of particular importance as the inability to properly metabolize carbohydrates represents the core dysfunction in diabetes. By preferentially converting energy production to carbohydrate metabolism, relative to lipids, diabetic patients avoid the consequences of elevated free fatty acids which invoke pro-inflammatory cytokines. In general, consistently elevated free fatty acids trigger a cascade of inflammatory processes which interfere with good health.

In sum, using the method of the present invention improves the carbohydrate processing system of the patient. This is quite important in diabetes. This is important and relevant in addressing a wide variety of other disease conditions.

The preferred embodiments described herein are illustrative only, and although the examples given include many specifications, they are intended as illustrative of only a few possible embodiments of the invention. Other embodiments and modifications will, no doubt, occur to those skilled in the art. The examples given should only be interpreted as illustrations of some of the preferred embodiments of the invention, and the full scope of the invention should be determined by the appended claims and their legal equivalents.

What is claimed is:

1. A method of stimulating cellular metabolism, comprising:
for a subject, having the subject consume a dose of carbohydrate, said dose sized at a selected amount per body weight, said dose providing more than 25% of the calories a healthy person of the subject's size should normally eat in a day,
dividing said dose into smaller portions and having the subject consume each smaller portion with some time between consuming subsequent smaller portions,
delivering insulin in a pattern and in an amount sufficient to induce cells to take up carbohydrate,
assessing the subject's blood glucose level and adjusting the insulin pattern amount and rate of insulin to balance the blood level of glucose to a physiologically acceptable range over the time period of the stimulation.

2. The method of claim 1 of stimulating cellular metabolism further comprising:
the dose of carbohydrate sized to provide between 33% and 55% of the calories a healthy person of the subject's size should normally eat in a day.

3. The method of claim 1 of stimulating cellular metabolism further comprising:
the dose of carbohydrate sized to provide more than 2.5 grams of carbohydrate per kilogram body weight of the subject.

4. The method of claim 1 of stimulating cellular metabolism further comprising:
the dose of carbohydrate sized to provide between about 3 and about 4 grams of carbohydrate per kilogram body weight of the subject.

5. The method of claim 1 of stimulating cellular metabolism further comprising:
dividing said dose into smaller portions administered over the course of a treatment day with the rate of glucose administration approximately averaged over the course of the insulin delivery.

6. The method of claim 5 of stimulating cellular metabolism further comprising:
dividing said dose into more than 5 smaller portions, administering a first smaller portion at a beginning time, then administering a second smaller portion at about a time which is a percentage of the planned treatment time approximately proportional to the cumulative dose administered before said second smaller dose.

7. The method of claim 6 of stimulating cellular metabolism further comprising:
the planned treatment time approximately proportional to the cumulative dose administered before said second smaller dose, where proportional is a ratio that can vary between 0.7 and 1.2 for % of total time over % cumulative dose.

8. The method of claim 5 of stimulating cellular metabolism further comprising:
dividing said dose into 9 smaller portions, administering a first smaller portion of about 10% of the planned total dose at a beginning time, then administering a second smaller portion of about 10% at about 30 minutes, then at about 30 minute intervals administering subsequent smaller portions of 13%, 10%, 10%, 13%, 10%, 10% and then a final balance of 13-14%.

9. The method of claim 1 of stimulating cellular metabolism further comprising:
delivering insulin in a pattern of microbursts.

10. The method of claim 9 of stimulating cellular metabolism further comprising:
delivering insulin in a pattern of microbursts where each microburst is equal to or greater than 10 mU/kg patient body weight, delivering a subsequent microburst within 4 to 7.5 minutes.

11. The method of claim 9 of stimulating cellular metabolism further comprising:
delivering insulin in a pattern of microbursts is delivered over a first period of time, followed by a rest period.

12. The method of claim 9 of stimulating cellular metabolism further comprising:
delivering insulin in a pattern of ten microbursts is delivered over a first period of approximately one hour.

13. The method of claim 9 of stimulating cellular metabolism further comprising:
delivering insulin in a pattern of microbursts is delivered in a pattern to keep blood glucose mostly in a range of 150 to 200 mg/dl.

14. The method of claim 13 of stimulating cellular metabolism further comprising:
delivering insulin in a pattern of microbursts that is increased in quantity, frequency, or both if the blood glucose goes above 200 mg/dl, and correspondingly decreased or stopped if the blood glucose goes below 150 mg/dl during a treatment day.

15. The method of claim 1 of stimulating cellular metabolism further comprising:
providing the carbohydrate in the form of an oral solution of dextrose.

16. The method of claim 1 of stimulating cellular metabolism further comprising:
assessing the subject's amount of carbohydrate metabolism at rest before starting treatment on a treatment day, and again after completing treatment on a treatment day.

17. The method of claim 16 of stimulating cellular metabolism further comprising:
scheduling a subsequent treatment day based on the subject's amount of carbohydrate metabolism during and after a treatment day.

18. The method of claim 16 of stimulating cellular metabolism further comprising:
scheduling a subsequent treatment day relatively sooner if the subject's amount of carbohydrate metabolism has not shown 35% improvement over the amount of carbohydrate metabolism before any treatment.

19. The method of claim 16 of stimulating cellular metabolism further comprising:
scheduling a subsequent treatment day relatively sooner if the subject's amount of carbohydrate metabolism has not shown a leveling off in improvement over a series of treatment days, indicative of approaching a maximum.

20. The method of claim 16 of stimulating cellular metabolism further comprising:
scheduling a subsequent treatment day in a pattern of two treatment days during a first week and a second week, then one treatment day per week, but increasing the number of treatment days per week if the subject's amount of carbohydrate metabolism has not shown a leveling off in improvement over a series of treatment days, indicative of approaching a maximum.

21. A method of stimulating cellular metabolism, comprising:
for a subject, having the subject consume a dose of dextrose, said dose sized at a selected amount per body weight, said dose providing more than 3 grams per kilogram of body weight,
dividing said dose into nine approximately equal smaller portions and having the subject consume each smaller portion at about thirty minute intervals,
delivering insulin in a pattern of microbursts with a time between microbursts of between 3.5 and 7 minutes, for a series of 8 to 12 microbursts in about 45 to 90 minutes, then taking a rest cycle, and continuing with a second series of microbursts in a similar pattern,
increasing said pattern of microbursts in quantity, frequency, or both if the blood glucose goes above 200 mg/dl, and correspondingly decreasing or stopping if the blood glucose goes below 150 mg/dl during a treatment day.
